(19) United States
(12) Patent Application Publication
(10) Pub. No.: US 2024/0238456 A1
(43) Pub. Date: Jul. 18, 2024

(54) SELF-ASSEMBLED BIOCOMPATIBLE IMAGING PARTICLES, THEIR SYNTHESIS AND THEIR USE IN IMAGING TECHNIQUES

(71) Applicants: INSTITUT NATIONAL DE LA SANTÉ ET DE LA RECHERCHE MÉDICALE, Paris (FR); UNIVERSITE DE CAEN NORMANDIE, Caen (FR); CENTRE HOSPITALIER UNIVERSITAIRE DE CAEN NORMANDIE, Caen (FR)

(72) Inventors: Maxime GAUBERTI, Caen (FR); Sara MARTINEZ DE LIZARRONDO, Caen (FR); Thomas BONNARD, Caed (FR); Charlène JACQMARCQ, Caen (FR); Denis VIVIEN, Caen (FR)

(21) Appl. No.: 18/291,677

(22) PCT Filed: Jul. 29, 2022
(86) PCT No.: PCT/EP2022/071456

§ 371 Date: Jan. 24, 2024

(30) Foreign Application Priority Data
Jul. 30, 2021 (EP) 21306073.4

(51) Int. Cl. A61K 49/18; A61K 49/16

(52) U.S. Cl. CPC A61K 49/1857; A61K 49/16; A61K 49/1887

(57) ABSTRACT

The present invention relates to a biocompatible particle comprising nanoparticles of iron oxide embedded in a polycathecolamine or polyserotonine matrix, a suspension of said particles, a process for preparing said suspension of particles, a conjugate comprising said particle and the use of said particle and said conjugate in imaging techniques.

The present invention relates to a biocompatible particle comprising nanoparticles of iron oxide embedded in a polycathecolamine or polyserotonine matrix, a suspension of said particles, a process for preparing said suspension of particles, a conjugate comprising said particle and the use of said particle and said conjugate in imaging techniques.

### BACKGROUND OF THE INVENTION

Magnetic resonance imaging (MRI) is a promising modality for molecular imaging but remains limited by a low sensitivity (Lohrke, J. et al., Adv. Ther., 2016, 33, 1-28). The usual concentration of relevant targets for molecular imaging is around 10⁻⁹-10⁻¹² M in human tissues, whereas MRI detects clinically approved gadolinium chelate at concentrations over 10⁻⁶ M. Amplification strategies aiming at binding a large number of contrast producing atoms to the molecular target are thus necessary. To date, nano-sized contrast agents such as ultrasmall particles of iron oxide (USPIO) with a diameter ranging from 10 to 50 nm have been the primary focus of molecular MRI studies (Laurent, S. et al., Chem. Rev., 2008, 108, 2064-2110). USPIO can be conjugated to targeting moieties such as peptides or antibodies and have a favorable safety profile in humans. However, the low sensitivity (due to the small amount of iron payload per particle), poor specificity (due to passive extravasation through permeated endothelial barriers) and long delay between administration and imaging (up to 24 hours after intravenous injection) have precluded the use of USPIO as targeted molecular imaging agents (Gauberti, M. et al., Front. Cell. Neurosci., 2014, 8, 389).

More recently, microparticles of iron oxide (MPIOs) with diameters close to one micrometer have been used as a new family of contrast agent for molecular MRI (McAteer, M. A. et al., Nat. Med., 2007, 13, 1253-1258). MPIOs display a higher sensitivity than USPIO thanks to a higher iron content (Gauberti, M. et al., Theranostics, 2018, 8, 1195-1212). Applicability of targeted MPIO for molecular MRI has been demonstrated in several experimental models, including cardiovascular (von zur Muhlen, C. et al., Circulation, 2008, 118, 258-267; von Zur Muhlen, C. et al., J. Clin. Invest., 2008, 118, 1198-1207) and neurovascular disorders (Quenault, A. et al., Brain, 2017, 140, 146-157), autoimmune diseases (Fournier, A. P. et al., Proc. Natl. Acad. Sci. U.S.A, 2017, 114, 6116-6121; Fournier, A. P. et al., Sci. Transl. Med., 2020, 12, eaaz4047) and cancer (Serres, S. et al., Proc. Natl. Acad. Sci. U.S.A, 2012, 109, 6674-6679). Notably, MPIOs are rapidly eliminated from the circulation by the reticulo-endothelial system, thereby limiting their ability to reach their target (Belliere, J. et al., Theranostics, 2015, 5, 1187-1202). Therefore, there are strict constrains on the construction and coating of MPIO to allow them to accumulate at concentrations high enough to be detected by MRI. Unfortunately, the MPIO used in preclinical studies are made of a polystyrene matrix and are not clinically compatible (Gauberti, M. et al., Front. Cell. Neurosci., 2014, 8, 389). Covalent assembly of USPIO with peptidase-degradable bonds has been described as an alternative to MPIO but the resulting product lacks sensitivity for molecular imaging (Perez-Balderas, F. et al., Nature communications, 2017, 8, 14254).

Thus, there is still no contrast agent combining the high sensitivity of currently available MPIO with the biocompatibility and biodegradability of USPIO.

### SUMMARY OF THE INVENTION

The inventors have now succeeded in developing a new type of contrast agent made of self-assembled submicrometric clusters of USPIO using a polycathecolamine or polyserotonine, in particular polydopamine (PDA), as an embedding matrix. Using only three reagents (iron chloride, a polycathecolamine or polyserotonine, and ammonia), submicrometric clusters of USPIO and polycathecolamine or polyserotonine with mean diameters ranging from 250 nm to 900 nm were produced. Thanks to the biocompatible, hydrophilic and reactive coating of polycathecolamine or polyserotonine (Lee, H. et al., Science, 2007, 318, 426-430; Wu, D. et al., Chem. Soc. Rev., 2021, 50, 4432-4483), the clusters of USPIO and polycathecolamine or polyserotonine can be efficiently functionalized with targeting moieties such as monoclonal antibodies (immuno-MRI). Such new platforms are useful for in vivo method of imaging, in particular ultra-sensitive molecular imaging of inflammation in the brain, kidneys and intestinal mucosa.

The invention therefore relates to a particle having a hydrodynamic diameter comprised between 200 and 2000 nm, said particle comprising nanoparticles of iron oxide embedded within a polymer matrix selected from polycathecolamines or polyserotonine.

The invention also relates to a suspension of said particles.

In another aspect, the present invention provides a process for preparing a suspension of particles of the invention, comprising the steps of:
a) Preparing a suspension of nanoparticles of iron oxide;
b) Coating of the nanoparticles of iron oxide with a catecholamine or serotonine;
c) Polymerizing the catecholamine or serotonine in the presence of the nanoparticles of iron oxide;
d) Terminating said polymerization; and
e) Recovering a suspension of particles.

The invention also relates to a conjugate comprising a particle of the invention or a suspension of particles of the invention and a molecule comprising free amine or thiol groups, in particular a monoclonal antibody.

The invention further relates to the particle of the invention, a suspension of particles of the invention or the conjugate of the invention for use in an in-vivo method of imaging.

The invention also relates to the use of the particles of the invention, a suspension of particles of the invention or the conjugate of the invention as a contrast agent or as a tracer in an imaging technique.

### DETAILED DESCRIPTION OF THE INVENTION

As detailed above, the invention relates to a particle having a hydrodynamic diameter comprised between 200 and 2000 nm, preferably between 250 and 1250 nm, more preferably between 300 and 1000 nm, still more preferably between 500 and 1000 nm, said particle comprising nanoparticles, preferably ultrasmall particles, of iron oxide embedded within a polymer matrix selected from polycathecolamines or polyserotonine.

As used herein, the expression “comprised between . . . and . . . ” should be understood to include the boundaries of the recited range. As used herein, the term “embedded” relative to nanoparticles and the surface of the polymer matrix refers to the nanaoparticles being at least partially extended into the surface such that the polymer is in contact with the nanoparticle to a greater degree than would occur if the nanoparticles were simply laid on the surface of the polymer matrix.

In the context of the invention, the term “hydrodynamic diameter” refers to the diameter of a hypothetical hard sphere that diffuses with the same speed as the particle being measured. It reflects the size of the particle when in solution and includes coatings or surface modifications made to the particle in question.

The hydrodynamic diameter of the particles of the invention may be determined according to any method known by the person skilled in the art. In particular, the hydrodynamic diameter of the particles of the invention may be determined by dynamic light scattering (DLS), with for example a NanoZS® apparatus (Malvern Instruments, Worcestershire, UK) equipped with a 633 nm laser at a fixed scattering angle of 173°, with the temperature of the cell being kept constant at 25° C. The particles are for this measure put in suspension in water at a concentration of 20 μg to 200 μg of iron per mL of water. Other known methods are particle tracking analysis (PTA) or its variant nanoparticle tracking analysis (PTA).

For example, the hydrodynamic diameter of the particle of the invention may be comprised between 200 and 2000 nm, between 200 and 1250 nm, between 200 and 1000 nm, between 200 and 900 nm, between 200 and 800 nm, between 200 and 700 nm, between 250 and 2000 nm, between 250 and 1250 nm, between 250 and 1000 nm, between 250 and 900 nm, between 250 and 800 nm, between 250 and 700 nm, between 300 and 2000 nm, between 300 and 1250 nm, between 300 and 1000 nm, between 300 and 900 nm, between 300 and 800 nm, between 300 and 700 nm, between 400 and 2000 nm, between 400 and 1250 nm, between 400 and 1000 nm, between 400 and 900 nm, between 400 and 800 nm, between 400 and 700 nm, between 500 and 2000 nm, between 500 and 1250 nm, between 500 and 1000 nm, between 500 and 900 nm, between 500 and 800 nm or between 500 and 700 nm.

Nanoparticles, preferably ultrasmall particles, of iron oxide incorporated in the particles of the invention can be chosen among maghemite of formula Fe₂O₃, magnetite of formula Fe₃O₄ or a mixture of Fe₂O₃ and Fe₃O₄. These different types of iron oxide are all superparamagnetic and biocompatible, allowing their use in particular as contrast agents in Magnetic Resonance Imaging (MRI) or as tracers in Magnetic Particle Imaging (MPI).

As used herein, the term “nanoparticle” refers to a material having a particle size of less than 1000 nm, in particular ranging from 1 to 500 nm, more particularly from 1 to 300 nm.

As used herein, the term “ultrasmall particle” refers to a material having a particle size of less than 50 nm, in particular ranging from 1 to 50 nm, more particularly from 1 to 30 nm.

In particular, the diameter of the nanoparticles, preferably ultrasmall particles, of iron oxide incorporated in the particles of the invention is comprised between 1 and 50 nm, more particularly between 1 and 30 nm.

For example, the diameter of the ultrasmall particles of iron oxide incorporated in the particles of the invention may be comprised between 1 and 45 nm, between 1 and 40 nm, between 1 and 35 nm, between 1 and 30 nm, between 2 and 50 nm, between 2 and 45 nm, between 2 and 40 nm, between 2 and 35 nm, between 2 and 30 nm, between 3 and 50 nm, between 3 and 45 nm, between 3 and 40 nm, between 3 and 35 nm, between 3 and 30 nm, between 5 and 50 nm, between 5 and 45 nm, between 5 and 40 nm, between 5 and 35 nm, between 5 and 30 nm, between 10 and 50 nm, between 10 and 45 nm, between 10 and 40 nm, between 1 and 35 nm or between 10 and 30 nm.

The polymer matrix of the particle of the invention is selected from polycathecolamines or polyserotonine.

In particular, the biodegradable polymer matrix in the particles of the invention can be selected from polydopamine (PDA), polynorepinephrine (PNE), polyepinephrine (PEP) and polyserotonine, more particularly from polydopamine (PDA) and polynorepinephrine (PNE). Still more particularly, the biodegradable polymer matrix in the particles of the invention is polydopamine (PDA).

In addition to their biodegradability, these different types of polymers have many advantages for the synthesis of the particles of the invention as well as their applications. In particular, a variety of ligand can be conjugated at high density with polycathecolamines or polyserotonine via Michael addition or Schiff base reactions (Lee, H. et al., Adv Mater. 2009, 21, 431-434). The ability to conjugate a large amount of targeting moieties on the surface of the USPIO clusters is required to maximize binding to the target and reach a high sensitivity. Third, polycathecolamines or polyserotonine are hydrophilic and negatively charged at physiological pH, providing a negative zeta-potential for the coated particles and preventing their aggregation in solution. They form a strong bond to iron oxide which allows to obtain stable particles even under sonication, allowing dispersing aggregated particles without breaking the clusters or detaching the conjugated ligands.

In one embodiment, the iron oxide concentration in the particle of the invention may be comprised between 50% and 95% in weight with respect to the total weight of the particle.

The iron concentration in the particle of the invention may be determined by any suitable known by the person skilled in the art. In particular, the iron concentration in the particle of the invention may be determined by the ferrozine method or by mass spectrometry.

The particles of the invention can be characterized by their polydispersity index. In one embodiment, the polydispersity index of the particle of the invention is below 0.3, in particular, the polydispersity index of the particle of the invention is comprised between 0.01 and 0.2.

The polydispersity index of the particle of the invention may be determined by any suitable known by the person skilled in the art. In particular, the polydispersity index of the particle of the invention may be determined by dynamic light scattering (DLS) by using for example the same apparatus and measurements conditions as those used for the measurement of the hydrodynamic diameter.

For example, the polydispersity index of the particle of the invention may be comprised between 0.01 and 0.3, between 0.02 and 0.3, between 0.03 and 0.3, between 0.05 and 0.3, between 0.07 and 0.3, between 0.1 and 0.3, between 0.01 and 0.2, between 0.02 and 0.2, between 0.03 and 0.2, between 0.05 and 0.2, between 0.07 and 0.2 or between 0.1 and 0.2.

The particles of the invention can also be characterized by their zeta-potential. In one embodiment, the zeta-potential of the particle of the invention is comprised between −50 and −20 mV, in particular between −45 and −25 mV, more particularly between −42 and −37 mV.

The zeta-potential may be determined by any suitable known by the person skilled in the art. In particular, the zeta-potential of the particle of the invention may be determined by electrophoretic light scattering (ELS) with a measurement carried out on the particles suspended in a 1 mM sodium chloride solution.

In the context of the invention, the term “zeta-potential” refers to the electrical potential at the interface which separates mobile fluid from fluid that remains attached to the surface of a particle.

Another object of the invention is a suspension of particles according to the invention. Such a suspension of particles contains a solvent which can be selected from an aqueous solution, for example water, or saline solution, or glycerol, or mannitol in which the particles of the invention described above are suspended.

In other words, the suspension of particles according to the invention comprises particles according to the invention suspended in a solvent selected from an aqueous solution, for example water, a saline solution, a glycerol solution and a mannitol solution. In particular, the suspension of particles according to the invention comprises particles according to the invention suspended in an aqueous solution or a saline solution. More particularly, the suspension of particles according to the invention comprises particles according to the invention suspended in an aqueous solution, preferably water, more preferably distilled water.

The mean hydrodynamic diameter of the particles in the suspension of particles according to the invention is comprised between 250 and 900 nm.

For example, the hydrodynamic diameter of the particles in the suspension of particles according to the invention may be comprised between 200 and 2000 nm, between 200 and 1250 nm, between 200 and 1000 nm, between 200 and 900 nm, between 200 and 800 nm, between 200 and 700 nm, between 250 and 2000 nm, between 250 and 1250 nm, between 250 and 1000 nm, between 250 and 900 nm, between 250 and 800 nm, between 250 and 700 nm, between 300 and 2000 nm, between 300 and 1250 nm, between 300 and 1000 nm, between 300 and 900 nm, between 300 and 800 nm, between 300 and 700 nm, between 400 and 2000 nm, between 400 and 1250 nm, between 400 and 1000 nm, between 400 and 900 nm, between 400 and 800 nm, between 400 and 700 nm, between 500 and 2000 nm, between 500 and 1250 nm, between 500 and 1000 nm, between 500 and 900 nm, between 500 and 800 nm or between 500 and 700 nm.

Advantageously, the mean hydrodynamic diameter of the particles in the suspension of particles according to the invention may be controlled.

In one embodiment, the mean hydrodynamic diameter of the particles in the suspension of particles of the invention is comprised between 250 and 400 nm, in particular between 250 and 350 nm, more particularly the mean hydrodynamic diameter of the particles of the invention is around 300 nm.

In another embodiment, the mean hydrodynamic diameter of the particles in the suspension of particles of the invention is comprised between 400 and 600 nm, in particular between 450 and 550 nm, more particularly the mean hydrodynamic diameter of the particles of the invention is around 500 nm.

In another embodiment, the mean hydrodynamic diameter of the particles in the suspension of particles of the invention is comprised between 600 and 900 nm, in particular between 650 and 750 nm, more particularly the mean hydrodynamic diameter of the particles of the invention is around 700 nm.

Another object of the invention is a process for preparing a suspension of particles of the invention, comprising the steps of:
a) Preparing a suspension of nanoparticles, preferably ultrasmall particles, of iron oxide, in particular having a diameter between 1 and 50 nm, more particularly between 1 and 30 nm;
b) Coating of the nanoparticles, preferably ultrasmall particles, of iron oxide with a catecholamine or serotonine, in particular with a catecholamine, more particularly with dopamine;
c) Polymerizing the catecholamine or serotonine in the presence of nanoparticles, preferably ultrasmall particles, of iron oxide;
d) Terminating said polymerization;
e) Recovering a suspension of particles.

The step a) of the process of the invention consists in preparing a suspension of nanoparticles, preferably ultrasmall particles, of iron oxide, in particular in water, more particularly in distilled water. In particular, the nanoparticles, preferably ultrasmall particles, of iron oxide may be chosen among maghemite of formula Fe₂O₃, magnetite of formula Fe₃O₄ or a mixture of Fe₂O₃ and Fe₃O₄. More particularly, the nanoparticles, preferably ultrasmall particles, of iron oxide are Fe₃O₄.

The step a) of the process of the invention may be performed according to any suitable method known by the person skilled in the art, including, for example, co-precipitation, solvothermal synthesis, thermal decomposition, polyol process, sonochemical reaction and sol-gel reaction. In particular, the step a) of the process of the invention may by performed by a co-precipitation method in an alkaline buffer. Typically, the co-precipitation method is performed by mixing FeCl₃ and FeCl₂, preferably in a molar ratio of 2:1, in an alkaline buffer, and adding progressively an ammonia solution, said ammonia solution being preferably at a concentration ranging from 10% to 15% (w/v), more preferably at a concentration of 13% (w/v), in particular at a rate ranging from 0.1 to 0.3 mL/min, more particularly from 0.15 to 0.25 mL/min, still more particularly at a rate of 0.2 mL/min, until precipitation of the iron oxide. The precipitate is optionally washed, preferably with distilled water, and resuspended, preferably in distilled water.

The step b) of the process of the invention consists in coating the iron oxide nanoparticles with a catecholamine or serotonine. Typically, the suspension of iron oxide nanoparticles obtained from step a) is mixed with a solution of catecholamine or serotonine, in particular a solution of catecholamine, more particularly a solution of dopamine, preferably in a weight ratio iron/catecholamine or serotonine comprised between 0.5 and 3.0, in particular between 0.5 and 2.5, more particularly between 1.0 and 2.0, still more particularly between 1.0 and 1.5, even more particularly a mass ratio iron/catecholamine or serotonine of 1.2.

After mixing the suspension of iron oxide nanoparticles obtained from step a) with a solution of catecholamine or serotonine, the step b) of the process of the invention may include a step of homogeneization of the resulting mixture, in particular by sonication, for example using a UP200ST sonicator (Hielscher) at 70% amplitude and 26 KHz, particularly during 1 to 30 minutes, more particularly 10 to 20 minutes, at a temperature ranging from 0° C. to 100° C., preferably 40° C. to 100° C.

The step b) of the process of the invention may further include a step of centrifugation, in particular at 3000G.

The step c) of the process of the invention consists in polymerizing the catecholamine or serotonine, in particular a catecholamine, more particularly dopamine, in which the iron oxide nanoparticles are coated.

The polymerization step may be performed by any suitable method known the skilled person in the art. In particular, the polymerization step may be performed by adding a base solution, preferably ammonia, and oxygen, preferably oxygen from the ambient air, to the ultrasmall particles of iron oxide coated with a catecholamine or serotonine obtained in step b) and stirring the resulting mixture during 15 minutes to 6 hours, preferably 30 minutes to 90 minutes, more preferably during 60 minutes. This leads to the self-assembly the iron oxide nanoparticles with the polycatecholamine or polyserotonine.

The amount of ammonia added during step c) allows to control the size of the particles of the invention.

In one embodiment, the ammonia added during step c) is comprised between 2.17 and 3.25 parts in weight with respect with 1 part of iron. Typically, between 2.0 mL and 3.0 mL of a 13% (w/v) ammonia solution is added for 120 mg of iron.

According to this embodiment, the mean hydrodynamic diameter of the particles is comprised between 250 and 400 nm, in particular between 250 and 350 nm, more particularly the mean hydrodynamic diameter of the particles is around 300 nm.

In another embodiment, the amount of ammonia added during step c) is comprised between 0.33 and 2.17 parts in weight with respect to 1 part of iron. Typically, between 0.3 mL and 2.0 mL of a 13% (w/v) ammonia solution is added for 120 mg of iron.

According to this embodiment, the mean hydrodynamic diameter of the particles is comprised between 400 and 600 nm, in particular between 450 and 550 nm, more particularly the mean hydrodynamic diameter of the particles is around 500 nm.

In another embodiment, the amount of ammonia added during step c) is comprised between 0.05 and 0.33 parts in weight with respect to 1 part of iron. Typically, between 0.05 mL and 0.3 mL of a 13% (w/v) ammonia solution is added for 120 mg of iron.

According to this embodiment, the mean hydrodynamic diameter of the particles is comprised between 600 and 750 nm, in particular between 650 and 750 nm, more particularly the mean hydrodynamic diameter of the particles is around 700 nm.

The step d) of the process of the invention consists in terminating the polymerization of step c).

The termination step may be performed by any suitable method known the skilled person in the art, such as, for example, the addition of an acid to get an acidic pH in the reaction mixture, the addition of a polymerization inhibitor, or replacing the reaction medium. In particular, the termination step is performed by replacing the reaction medium. Typically, the particles are separated from the solution of base in which the polymerization step c) was performed, in particular using a separating magnet or a centrifugation step, more particularly a centrifugation step, allowing to keep the particles in a bottom layer and the base solution as a supernatant. The supernatant is then removed and replaced by a wash solution, in particular water, more particularly distilled water. This operation can be done multiple times to ensure that the base solution from step c) is totally removed and replaced by the wash solution.

The step e) of the process of the invention consists in the recovery of a suspension of the particles of the invention. These particles can then be stored at a low temperature of about 3 to 10° C., either in water, or saline solution.

Prior to its injection to a patient, the suspension of particles of the invention is kept under agitation at a temperature of 0 to 37° C., in particular 4° C.

Another object of the invention is a suspension of particles or a particle obtained by the process according to the invention.

Another object of the invention is a particle obtained by the process according to the invention after an additional step of isolation of said particle by removal of the solvent of the suspension obtained after step e).

The present invention also relates to a conjugate comprising a particle of the invention or a suspension of particles of the invention and a molecule comprising free amine or thiol groups.

In particular, the molecule comprising free amine or thiol groups is chosed from a protein, a peptide, a nanobody, a monoclonal antibody, or a molecule comprising a radiolabeled metal. Preferably, the molecule comprising free amine or thiol groups is a monoclonal antibody.

In an embodiment, the conjugate comprises a particle of the invention or a particle obtained by the process of the invention and a molecule comprising at least a free amine or a thiol group. In particular, the molecule comprising at least a free amine or a thiol group a protein may be a peptide, a nanobody, a monoclonal antibody, a molecule comprising a radiolabeled metal or a small molecule, such as for example N-acetyl cysteine. More particularly, the molecule comprising at least a free amine or a thiol group a protein may be a peptide, a nanobody, a monoclonal antibody, a molecule comprising a radiolabeled metal or N-acetyl cysteine. Even more particularly, the molecule comprising at least a free amine or a thiol group a protein may be a peptide, a nanobody or a monoclonal antibody. Preferably, the molecule comprising at least a free amine or a thiol group a protein may be a monoclonal antibody.

In an embodiment, the molecule comprising at least a free amine or a thiol group may be a molecule modified with a linker comprising at least a free amine or a thiol group. In other words, the molecule comprising at least a free amine or a thiol group is a molecule wherein the free amine or thiol group is held by a linker moiety.

In the context of the invention, the term “linker” or “linker moiety” refers to a connector for linking a molecule to a particle of the invention or a particle obtained by the process of the invention.

In the context of the invention, the term “conjugate” refers to a molecule composed of two or more molecules which are linked together. The conjugates of the invention are typically composed of a particle according to the invention linked to a protein, a peptide, a nanobody, or a monoclonal antibody. The particles of the invention can also be linked to radiolabeled metals, such as Copper⁶⁴, or Gallium⁶⁸, in particular in the context of a use in Positron Emission Tomography (PET) imaging technique.

Particularly, the monoclonal antibody may be chosen from immunoglobulin G (IgG), vascular-cell adhesion molecule 1 (VCAM-1), intercellular adhesion molecule 1 (ICAM-1), P-Selectin, E-Selectin or mucosal addressin cell adhesion molecule 1 (MAdCAM-1). More particularly, the monoclonal antibody may be chosen from vascular-cell adhesion molecule 1 (VCAM-1), intercellular adhesion molecule 1 (ICAM-1) or mucosal addressin cell adhesion molecule 1 (MAdCAM-1). Still more particularly, the monoclonal antibody may be vascular-cell adhesion molecule 1 (VCAM-1) or mucosal addressin cell adhesion molecule 1 (MAdCAM-1).

In one embodiment, the monoclonal antibody may be chosen from vascular-cell adhesion molecule 1 (VCAM-1), intercellular adhesion molecule 1 (ICAM-1), P-Selectin, E-Selectin or mucosal addressin cell adhesion molecule 1 (MAdCAM-1). More particularly, the monoclonal antibody may be chosen from vascular-cell adhesion molecule 1 (VCAM-1), intercellular adhesion molecule 1 (ICAM-1) or mucosal addressin cell adhesion molecule 1 (MAdCAM-1). Still more particularly, the monoclonal antibody may be vascular-cell adhesion molecule 1 (VCAM-1) or mucosal addressin cell adhesion molecule 1 (MAdCAM-1).

Particularly, the molecule comprising at least a free amine or thiol group can be a fibrinolytic agent. In an embodiment, the fibrinolytic agent is bound to the particle of the invention, or to a particle obtained by the process of the invention, by a linker moiety having at least a free amine or thiol group.

Particularly, the molecule comprising at least a free amine or a thiol group is a protein that can be a tissue plasminogen activator or a fragment thereof, for example a recombinant tissue plasminogen activator (rtPA) such as alteplase, reteplase, or tenecteplase. In an embodiment, the protein that can be a tissue plasminogen activator or a fragment thereof is bound to the particle of the invention, or the particle obtained by the process of the invention by a linker moiety having at least a free amine or thiol group.

The amount of particle according to the invention in the conjugate may be comprised between 50% and 99%, in particular between 65% and 95%, more particularly between 80% and 95%, still more particularly between 85% and 95%, in weight with respect to the total weight of the conjugate.

After their preparation, the conjugates of the invention are kept under agitation at a temperature of 0 to 37° C., in particular 4° C., until their injection to a patient.

In order to be used in an in vivo method of imaging, the particles of the invention or obtained by the process of the invention or the conjugate of the invention have to be administered to a patient prior to the imaging step. The particle of the invention or the conjugate of the invention may be administered as a formulation in an effective amount by any of the accepted modes of administration, preferably by intravenous, intraarterial or oral route.

Another object of the invention is therefore the particle of the invention, the suspension of particles of the invention, the particle or the suspension of particles obtained by the process of the invention or the conjugate of the invention for use in an in vivo method of imaging.

The particles of the invention, the suspension of particles of the invention, the particle or suspension of particles obtained by the process of the invention or the conjugates of the invention have plural applications in the imaging field.

In particular, the particles of the invention, the suspension of particles of the invention, the particle or suspension of particles obtained by the process of the invention or the conjugates of the invention can be used in imaging techniques such as Magnetic Resonance Imaging (MRI), Magnetic Particle Imaging (MPI), photoacoustic imaging, or Positron Emission Tomography (PET). More particularly, they can be used in Magnetic Resonance Imaging (MRI).

In the context of photoacoustic imaging, the absorption spectrum of the particles of the invention are particularly adapted to get a good visualization. Indeed, polydopamine for example absorbs mostly in the near infrared (wavelengths around 700 nm) and is well distinguished from oxygenated hemoglobin which absorbs mostly in the far infrared (wavelengths around 900 nm).

In the context of Positron Emission Tomography (PET), the particles of the invention or the particles obtained by the process of the invention can be used after a conjugation and/or a radiolabeling step. Radiolabeled metals can then be used, such as Copper⁶⁴, or Gallium⁶⁸.

All these techniques can be used for in vivo diagnostic. The mode of action of the particles of the invention make them suitable for all types of imaging. In particular, the conjugate of the invention can used in the molecular imaging of inflammation in the brain, heart, lungs, kidneys and intestinal mucosa, more particularly in the molecular imaging of inflammation in the brain, kidneys and intestinal mucosa.

The present invention is also directed to an in vivo diagnostic method using the particles of the invention, a suspension of particles of the invention, the particles or suspension of particles obtained by the process of the invention or the conjugate of the invention.

The invention also provides a composition containing the particles of the invention, a suspension of particles of the invention, the particles or suspension of particles obtained by the process of the invention or a conjugate of the present invention. Said composition may further comprise at least one pharmaceutically acceptable carrier, diluent, excipient and/or adjuvant.

Such a composition can contain particles of the invention, a suspension of the particles of the invention, the particles or suspension of particles obtained by the process of the invention, or conjugates of the invention in a solvent, such as for example a solution of mannitol 0.3 M.

Prior to their injection, the particles of the invention can be suspended in any physiological medium compatible with an injection to a human patient. Examples of such physiological medium are mannitol or glycerol, in particular a mannitol solution or a glycerol solution, more particularly a 0.3 M mannitol solution.

Another object of the invention is a method of imaging wherein a patient has been administered, for example by injection, a composition containing the particles of the invention, a suspension of particles of the invention, the particles or suspension of particles obtained by the process of the invention or a conjugate of the present invention and comprising an imaging step.

Another object of the invention is the use of the particles of the invention, a suspension of the particles of the invention, the particles or suspension of particles obtained by the process of the invention or the conjugate of the present invention as a contrast agent or as a tracer in an imaging technique.

### Definitions

The definitions and explanations below are for the terms as used throughout the entire application, including both the specification and the claims.

When describing the particles of the invention, the terms used are to be construed in accordance with the following definitions, unless indicated otherwise.

The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

The term “biodegradable” as used herein refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers in the context of the present invention include, for example, polydopamine (PDA), polynorepinephrine (PNE), polyepinephrine (PEP) and polyserotonine (PST), polydopamine (PDA) being particularly preferred.

As used herein, the term “embedded” relative to nanoparticles and the surface of the polymer matrix refers to the nanaoparticles being at least partially extended into the surface such that the polymer is in contact with the nanoparticle to a greater degree than would accur if the nanoparticles were simply laid on the surface of the polymer matrix.

The term “suspension” as used herein refers to a heterogeneous mixture of materials comprising a liquid and a finely dispersed solid material.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The term “administration”, or a variant thereof (e.g., “administering”), means providing the active agent or active ingredient, alone or as part of a pharmaceutically acceptable composition, to the patient in whom/which the condition, symptom, or disease is to be treated.

By “pharmaceutically acceptable” is meant that the ingredients of a pharmaceutical composition are compatible with each other and not deleterious to the patient thereof.

The term “excipient” as used herein means a substance formulated alongside the active agent or active ingredient in a pharmaceutical composition or medicament. Acceptable excipients for therapeutic use are well known in the pharmaceutical art, and are described, for example, in Remington's Pharmaceutical Sciences, 21^{st} Edition 2011. The choice of excipient can be selected with regard to the intended route of administration and standard pharmaceutical practice. The excipient must be acceptable in the sense of being not deleterious to the recipient thereof. The at least one pharmaceutically acceptable excipient may be for example, a binder, a diluent, a carrier, a lubricant, a disintegrator, a wetting agent, a dispersing agent, a suspending agent, and the like.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The present invention will be better understood with reference to the following examples and figures. These examples are intended to be representative of specific embodiments of the invention, and are not intended as limiting the scope of the invention.

### FIGURES

FIG. 1. Representative phase-microscopy images of USPIO(n)@PDA of different sizes, obtained by using different concentrations of ammonia during synthesis.

FIG. 2. Size distribution of USPIO(n)@PDA as assessed by dynamic light scattering (representative of 3 independent experiments in three independent batches).

FIG. 3A-H. Relaxivity measurement of large 700 nm USPIO₍ₙ₎@PDA using a 7T preclinical MRI: (a) Hysteresis cycles at 5K of small (left), medium (middle) and large (right) USPIO(n)@PDA clusters; (b) Hysteresis cycles at 300K of small (left), medium (middle) and large (right) USPIO(n)@PDA clusters; (c) Summary of the results. Hc: coercive field. Ms: saturation magnetization.

FIG. 4A-D. Degradation of large USPIO(n)@PDA clusters in different buffers as assessed by visual inspection and UV-Visible spectroscopy: UV-Vis spectroscopy of the large USPIO(n)@PDA clusters (700 nm) in PBS (a), ALF (v), citrate (c) and citrate with H₂O₂(d) at different time points.

FIG. 5A-C. Degradation of large USPIO(n)@PDA clusters after 7 days of incubation in citrate buffer with hydrogen peroxide: UV-Vis spectroscopy of the large USPIO₍ₙ₎ @PDA clusters (700 nm) and USPIO@Dopamine in PBS (b), citrate (c) and citrate with H₂O₂(d).

FIG. 6A-C. T2-weighted imaging after intravenous injection of 4 mg/kg large USPIO₍ₙ₎@PDA: (a) Evolution of the signal intensity on T2-weighted imaging in the liver (n=7 per group). Ratio was calculated by taking the signal of the paravertebral muscles as reference; (b) Evolution of the signal intensity on T2-weighted imaging in the spleen (n=7 per group); (c) Evolution of the signal intensity on T2-weighted imaging in the right kidney (n=7 per group). * p<0.05 versus baseline.

FIG. 7A-D. Cytotoxicity of large USPIO@PDA clusters on human venous endothelial cells (HUVEC): (a-b) Viability of HUVEC cells 3 hours (a) and 24 hours (b) after incubation with different concentrations of large USPIO@PDA clusters as assessed by the lactate dehydrogenase (LDH) assay (n=5 per group); (c-d) Viability of HUVEC cells 3 hours (c) and 24 hours (d) after incubation with different concentrations of large USPIO@PDA clusters as assessed by the Water Soluble Tetrazolium-1 (WST-1) assay (n=5 per group). *p<0.05 compared to the control group (0 μg/ml).

FIG. 8A-C. Hemolysis assays in mouse and human blood: (a) Spectroscopy of the supernatant of test tubes containing mouse blood after incubation with different concentrations of large USPIO₍ₙ₎@PDA clusters and centrifugation; (b) Spectroscopy of the supernatant of test tubes test tubes containing human blood after incubation with different concentrations of large USPIO₍ₙ₎@PDA clusters and centrifugation; (c) Summary of the hemolysis rate for each conditions measured by absorbance at 540 nm (representative of n=3 per group). No condition exceeded 5% of hemolysis.

FIG. 9A-B. Rotational thromboelastometry (ROTEM) in the presence of large USPIO₍ₙ₎@PDA clusters in whole human blood: (a) Representative ROTEM curves using the EXTEM assay with different concentrations of large USPIO@PDA clusters; (b) Corresponding main ROTEM parameters, showing no significant effect of large USPIO@PDA clusters on clot formation.

FIG. 10A-C. Clot lysis assay using human plasma in the presence of large USPIO₍ₙ₎@PDA clusters: (a) Representative clot lysis assay curves showing how 75% clotting time (75% CT) and 50% lysis time (50% LT) were measured; (b) Mean 75% CT of human plasma in the presence of different concentrations of large USPIO₍ₙ₎@PDA clusters (n=5 per group); (c) Mean 50% LT of human plasma in the presence of different concentrations of large USPIO₍ₙ₎@PDA clusters (n=5 per group). The 50% LT was set to 350 minutes in samples without lysis.

FIG. 11A-C. Clot lysis assay using mouse plasma in the presence of large USPIO₍ₙ₎@PDA clusters: (a) Representative clot lysis assay curves showing how 75% clotting time (75% CT) and 50% lysis time (50% LT) were measured; (b) Mean 75% CT of mouse plasma in the presence of different concentrations of large USPIO₍ₙ₎@PDA clusters (n=5 per group); (c) Mean 50% LT of mouse plasma in the presence of different concentrations of large USPIO₍ₙ₎@PDA clusters (n=5 per group). The 50% LT was set to 350 minutes in samples without lysis.

FIG. 12. Body weight of mice at baseline and at different time points after intravenous injection of large USPIO₍ₙ₎@PDA clusters (4 mg/kg).

FIG. 13. Plasma concentrations of liver enzymes at different time points after intravenous injection of large USPIO₍ₙ₎@PDA clusters (4 mg/kg) in mice as assessed by ELISA. The negative control (Negative Ctrl) samples were collected in control mice that did not receive USPIO(n)@PDA. The positive control (Positive Ctrl) samples were collected 24 hours after intra-peritoneal injection of 5 mg/kg E. coli lipopolysaccharide (LPS). * p<0.05 versus negative control. ALAT: Alanine transaminase. ASAT: Aspartate aminotransferase.

FIG. 14A-F. Plasma concentrations of a set of key cytokines and chemokines at different time points after intravenous injection of large USPIO₍ₙ₎@PDA clusters (4 mg/kg) in mice as assessed by ELISA. The negative control (Negative Ctrl) samples were collected in control mice that did not receive USPIO(n)@PDA. The positive control (Positive Ctrl) samples were collected 24 hours after intra-peritoneal injection of 5 mg/kg E. coli lipopolysaccharide (LPS). * p<0.05 versus negative control. TNF: tumor necrosis factor. IL: Interleukin. IFN: interferon. CXCL1: chemokine (C—X—C motif) ligand 1.

FIG. 15A-C. Complexation of immunoglobulins to USPIO(n)@PDA: (a) UV-Vis spectroscopy of the supernatant of USPIO₍ₙ₎@PDA@IgG after complexation to different doses of IgG (from 80 to 400 μg/mg); (b) SDS-PAGE of the supernatant of USPIO₍ₙ₎@PDA@IgG after complexation to different doses of IgG; (c) Flow-cytometry of USPIO₍ₙ₎@PDA@IgG after complexation to different doses of IgG (rat) using anti-rat or control (anti-goat) secondary antibodies.

FIG. 16. Characterization of large USPIO₍ₙ₎@PDA clusters after complexation to monoclonal antibodies targeting mouse vascular cell adhesion molecule-1 (VCAM-1): Quantification of particle fluorescence according to the type of fluorescent secondary antibodies (n=25 per group).

FIG. 17. Quantification of the number of particles per field (n=5 per group) in anti-human USPIO₍ₙ₎@PDA@αVCAM-1 or control USPIO₍ₙ₎@PDA@IgG after incubation with quiescent (treated with PBS) or activated (treated with TNF) human endothelial cells (HCMECD/3). * p<0.05 versus all other groups.

FIG. 18A-B. Human brain endothelial cells (HCMECD/3) overexpress VCAM-1 after stimulation with TNF as assessed by flow cytometry: (a) Left: Flow cytometry results using primary anti-VCAM-1 antibody or control immunoglobulin (IgG) after stimulation with TNF (50 ng/ml for 24 hours). Right: corresponding density plot; (b) Same as in (a) in unstimulated cells.

FIG. 19. Quantification of USPIO₍ₙ₎@PDA@αVCAM-1 induced signal void in the right striatum (n=5) after successive intravenous injection of USPIO₍ₙ₎@PDA targeted against VCAM-1 using monoclonal antibodies (USPIO₍ₙ₎@PDA@αVCAM-1) up to 4 mg/kg (equivalent iron) 24 hours after injection of 1 μg LPS in the right striatum.

FIG. 20. Quantification of USPIO₍ₙ₎@PDA@αVCAM-1 induced signal void in the right striatum (n=5 per group) after intravenous injection of 4 mg/kg USPIO₍ₙ₎@PDA@αVCAM-1 24 hours after injection of different doses of LPS in the right striatum (from 0 to 1.0 μg).

FIG. 21. Quantification of signal void in the right striatum (n=5 per group) after intravenous injection of 4 mg/kg USPIO₍ₙ₎@PDA@IgG or USPIO₍ₙ₎@PDA@αVCAM-1 24 hours after injection of 1 μg of LPS in the right striatum.

FIG. 22. USPIO₍ₙ₎@PDA@αVCAM-1 accumulate in the inflamed striatum of LPS treated mice: Quantification (n=4 per group) in the striatum of mice at 24 hours after intrastrial LPS injection (1.0 μg) and 60 minutes after intravenous injection of USPIO₍ₙ₎@PDA@αVCAM-1. Three images from three different animals are presented for USPIO₍ₙ₎ @PDA@αVCAM-1 and for control USPIO₍ₙ₎@PDA@IgG.

FIG. 23. Higher density of anti-VCAM-1 monoclonal antibodies on the surface of USPIO₍ₙ₎@PDA improves the sensitivity of molecular MRI: Quantification (n=4 per group) in the brain after intrastriatal injection of LPS (1.0 μg) and after intravenous injection of USPIO₍ₙ₎@PDA (4 mg/kg) with either 100% IgG on their surface (top), 50% control IgG and 50% anti-VCAM-1 antibodies (middle) or 100% anti-VCAM-1 antibodies.

FIG. 24. Saturation of USPIO₍ₙ₎@PDA@αVCAM-1 binding sites with free anti-VCAM-1 monoclonal antibodies prevent USPIO₍ₙ₎@PDA@αVCAM-1 binding in the inflamed brain: Quantification of USPIO₍ₙ₎@PDA@αVCAM-1 induced signal voids (n=4 per group) in the brain after intrastriatal injection of LPS (1.0 μg) both before and after intravenous injection of USPIO₍ₙ₎@PDA@αVCAM-1 (4 mg/kg) in mice that received an intravenous injection of 100 μg of either control immunoglobulin (left) or anti-VCAM-1 monoclonal antibodies (right) 15 minutes before imaging.

FIG. 25A-B. Analysis of the binding kinetic of USPIO₍ₙ₎@PDA@IgG and USPIO₍ₙ₎@PDA@αVCAM-1 in the LPS model of neuroinflammation: (a) Longitudinal evolution of the MRI signal in the right striatum (orange) and in the ophthalmic vein (blue) after intravenous injection of USPIO₍ₙ₎@PDA@IgG 24 hours after intrastriatal injection of LPS (1.0 μg); (b) Longitudinal evolution of the MRI signal in the right striatum (orange) and in the ophthalmic vein (blue) after intravenous injection of USPIO₍ₙ₎@PDA@αVCAM-1 24 hours after intrastriatal injection of LPS (1.0 μg).

FIG. 26. Longitudinal follow-up of USPIO₍ₙ₎@PDA@αVCAM-1 induced signal voids reveals progressive unbinding of the particles: quantification (n=3 mice at each time points) in the brain after intrastriatal injection of LPS (1.0 μg) both before and at different time points after a single intravenous injection of USPIO₍ₙ₎@PDA@αVCAM-1 (4 mg/kg).

FIG. 27. Unbound USPIO₍ₙ₎@PDA@IgG accumulate in the macrophages of the liver: Quantification (n=4 per group) in the liver of mice 60 minutes after intravenous injection of USPIO₍ₙ₎@PDA@IgG (4 mg/kg) or control mice. USPIO₍ₙ₎@PDA@IgG are revealed by fluorescent secondary anti-rat antibodies.

FIG. 28. Clustering USPIO₍ₙ₎@PDA into submicrometric particles improves sensitivity of molecular MRI of VCAM-1: Quantification (n=4 per group) after intravenous injection of 4 mg/kg of the particles 24 hours after injection of 1 μg of LPS in the right striatum.

FIG. 29A-C. Molecular imaging of endothelial activation in clinically relevant experimental models. (a) Corresponding quantification of signal void in the right hemisphere (n=5 per group) after intravenous injection of 4 mg/kg USPIO₍ₙ₎@PDA@αVCAM-1 (left) or USPIO₍ₙ₎@PDA@IgG (right) 24 hours after ischemic stroke induction by electrocoagulation of the right middle cerebral artery; (b) Corresponding quantification of signal void in the kidney medulla (n=5 per group) after intravenous injection of 4 mg/kg USPIO₍ₙ₎@PDA@αVCAM-1 (left) or USPIO₍ₙ₎@PDA@IgG (right) 48 hours after acute kidney injury (rhabdomyolysis) induced by intramuscular injection of 50% glycerol; (c) Corresponding quantification of signal void in the descending colon (n=5 per group) after intravenous injection of 4 mg/kg USPIO₍ₙ₎@PDA@αMAdCAM-1 (left) or USPIO₍ₙ₎ @PDA@IgG (right) in an acute colitis model induced by 5 day treatment with 2.0% of dextran sodium sulfate in the drinking water.

### EXAMPLES

### Abbreviations

DSS: dextran sulfate sodium
IgG: immunoglobulin G
LPS: lipopolysaccharide
MAdCAM-1: mucosal addressin cell adhesion molecule 1
MRI: magnetic resonance imaging
MPIO: microparticles of iron oxide
PBS: phosphate buffered saline
PDA: polydopamine
USPIO: ultrasmall particles of iron oxide
VCAM-1: vascular-cell adhesion molecule 1

### Materials and methods

### Reagents

The following reagents were purchased from Sigma-Aldrich: ferric chloride hexahydrate, ferrous chloride tetrahydrate, ammonia solution, dopamine hydrochloride, sodium phosphate monobasic, sodium phosphate dibasic, mannitol. Commercial microparticles of iron oxide (MPIO; diameter 1.08 μm) with COOH surface groups were purchased from Fisher Technology.

### Synthesis of Ultrasmall Particles of Iron Oxide (USPIO)

USPIO were produced by a co-precipitation method in an alkaline buffer. In a typical synthesis, 540 mg of FeCl₃·6H₂O and 198.8 mg of FeCl₂·4H₂O were dissolved in 5.7 mL of distilled water by vortexing, yielding a homogenous yellow solution. Under continuous agitation at room temperature, 6.3 mL of a 13% (w/v) ammonia solution was progressively added at a rate of 0.2 mL/min. The solution turned from yellow to brown and ultimately to a deep black color, corresponding to the formation of magnetite. The precipitate was washed 5 times with distilled water by magnetic separation and resuspended in 10 mL of distilled water.

Synthesis of Ultrasmall Particles of Iron Oxide Coated with Dopamine (USPIO@Dopamine)

Eight milliliter of the solution of USPIO was resuspended in 40 mL of a solution containing 2.5 mg/mL of dopamine hydrochloride. The resulting solution was sonicated for 15 minutes at 70% amplitude and 26 KHz using a UP200ST sonicator (Hielscher). The color of the solution slightly changed from black to dark brown. Then, the solution was centrifuged at 3000G for 5 minutes to remove large remaining USPIO aggregates. 30 mL of the supernatant containing USPIO@Dopamine and free dopamine hydrochloride were transferred to a new vial.

Synthesis of Particles of the Invention Comprising Ultrasmall Particles of Iron Oxide Embedded within Polydopamine (USPIO₍ₙ₎@PDA)

The USPIO@Dopamine/Dopamine hydrochloride solution were placed under vigorous steering using an Ultra-Turrax T-25 disperser at 20.500 rpm. To produce large USPIO₍ₙ₎@PDA, 67 μL of a 13% (w/v) ammonia solution was first added to the solution which was left to react for 60 minutes. Then, 203 μL of a 13% (w/v) ammonia solution was added and the incubation was continued for 30 minutes to allow further polymerization of dopamine. To produce medium sized USPIO(n)@PDA, 270 μL of a 13% (w/v) ammonia solution was added in one time and the solution was left to react for 60 minutes. To produce small sized USPIO(n)@PDA, 2160 μL of a 13% (w/v) ammonia solution was added in one time and the solution was left to react for 60 minutes. Then, the solution was centrifuged at 1000G for 3 minutes to remove the largest aggregates, the pellet was discarded and 24 mL of the supernatant were transferred to a new vial. The USPIO₍ₙ₎@PDA were then washed five-time with distilled water and finally resuspended in 8 mL of distilled water and stored at 4° C. until further use.

### Determination of the Hydrodynamic Diameter of the Particles of the Invention

Dynamic light scattering (DLS) was used to determine the average hydrodynamic diameter, the polydispersity index (PDI) and the diameter distribution by volume of the USPIO₍ₙ₎@PDA particles with a NanoZS@ apparatus (Malvern Instruments, Worcestershire, UK) equipped with a 633 nm laser at a fixed scattering angle of 173°. The temperature of the cell was kept constant at 25° C. and all dilutions were performed in pure water. The particles are for this measure put in suspension in water at a concentration of 20 μg to 200 μg of iron per mL of water. Measurements were performed in triplicate.

### Determination of Iron Concentration in the Particles of the Invention

Iron content of USPIO₍ₙ₎@PDA suspension was measured with FerroZine method. Particles were degraded overnight at room temperature in HCl 1M, releasing ferric (Fe³⁺) and ferrous (Fe²⁺) ions in solution. Samples were incubated 30 min with ascorbic acid 0.65% (w/v) to reduce ferric ions in ferrous ions. Samples pH was adjusted with ammonium acetate 12% (w/v). 3-(2-Pyridyl)-5,6-diphenyl-1,2,4-triazine-p,p′-disulfonic acid monosodium salt hydrate was added (FerroZine Iron Reagent, Sigma-Aldrich, 1 mM) and absorbance was measured at 562 nm with a spectrophotometer (ELx808 Absorbance reader, BioTeK) indicating the amount of complexes formed with ferrous ions. Iron content was determined against standard curves obtained from iron chloride dilutions.

Coating of Particles of the Invention (USPIO₍ₙ₎@PDA) with Antibodies

In a typical coating procedure, 2.8 mg of USPIO(n)@PDA were washed one time with purified water and resuspended in 5 mL of 10 mM phosphate buffer (pH 8.5). Then, 400 μg of monoclonal antibodies (or another appropriate amount) were incubated with USPIO(n)@PDA at room temperature for 24 hours. The resulting solution was sonicated for 5 minutes at 20% amplitude and 26 KHz using a UP200ST sonicator to break any aggregates. The coated USPIO(n)@PDA were then washed three times with a 0.3 M mannitol solution and finally resuspended in 5 mL of a 0.3 M mannitol solution and stored at 4° C. until further use.

### Measurements of the Relaxivities by 1H 7T MRI.

Large 700 nm USPIO(n)@PDA were dispersed in agarose gels (2%) in Tris-Acetate-EDTA buffer at different concentrations. The sample were then imaged using a BioSpec 7 T TEP-MRI and the following sequences were performed: T1 mapping using Flow-sensitive Alternating Inversion Recovery (FAIR)—RARE sequence with repetition time (TR)=3000 ms and inversion Time (TI) ranging from 6.5 ms to 2000 ms; T2 mapping using Multislice Multiecho (MSME) sequence with TR=4000 ms and echo time (TE) ranging from 3.65 to 51.11 ms; T2* mapping using multi gradient echo (MGE) sequence with TR=4000 ms and TE ranging from 2 ms to 17.47 ms. The corresponding R1, R2 and R2* relaxivities were calculated as described above.

### Animals

All experiments were performed on 8 to 16-week-old male Swiss mice (Janvier, France). Animals were maintained under specific pathogen-free conditions at the Centre Universitaire de Ressources Biologiques (CURB, Basse-Normandie, France) and all had free access to food and tap water.

### Magnetic Resonance Imaging (MRI) In Vivo

Experiments were carried out on a Pharmascan 7 T/12 cm system using surface coils (Bruker, Germany). Mice were anesthetized with isoflurane (1.5%-2.0%), maintained at 37° C. by the integrated heat animal holder and the breathing rate was monitored during the imaging procedure. T2-weighted images were acquired using a MSME sequence: TE/TR 51 ms/2500 ms with 70 μm*70 μm*500 μm spatial resolution. T2*-weighted 3D fast low angle shot gradient echo imaging with flow compensation (FLASH, spatial resolution of 78 μm*78 μm*150 μm) with TE/TR 8.6 ms/50 ms and a flip angle (FA) of 200 was performed to reveal USPIO₍ₙ₎@PDA clusters and USPIO (acquisition time=17 min). High resolution T2*-weighted images presented in this study are minimum intensity projections of 3 consecutive slices (yielding a Z resolution of 450 μm).

### Statistical Analysis

Results are presented as the mean±SD. Statistical analyses were performed using Mann-Whitney's U-test. When more than two groups were compared, statistical analyses were performed using Kruskal-Wallis (for multiple comparisons) followed by post-hoc Mann-Whitney's U-test. When comparing two groups, a p-value <0.05 was considered significant (two sided).

### Results

### Synthesis of USPIO₍ₙ₎@PDA Submicrometric Clusters

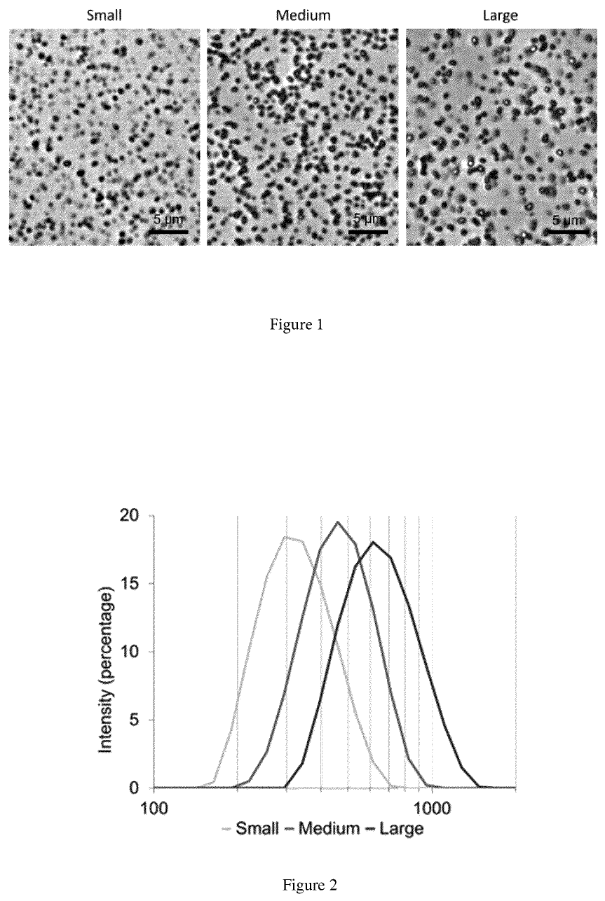

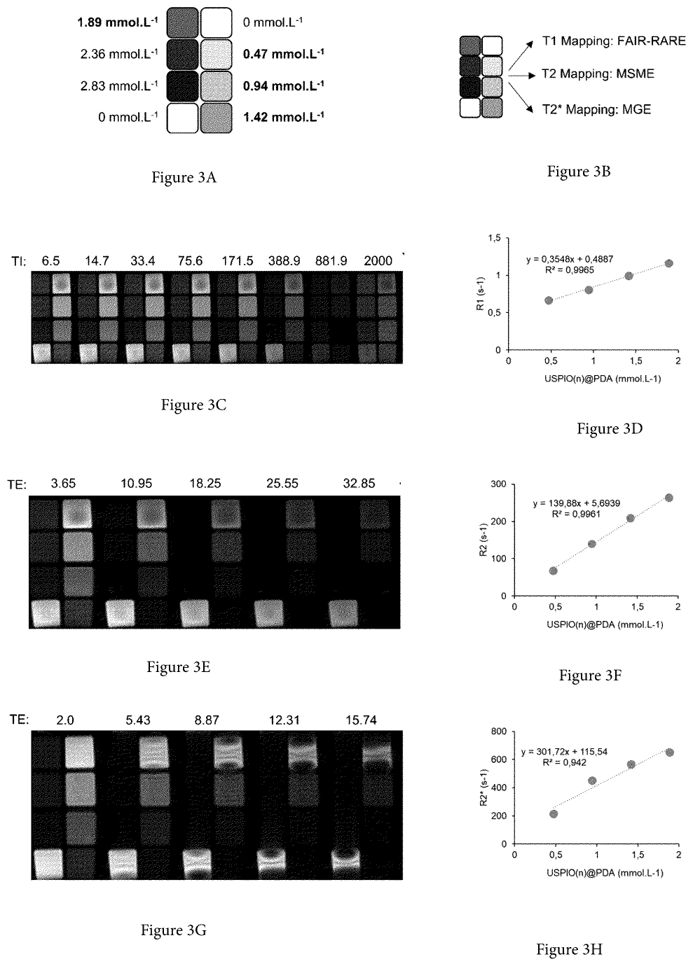

In alkaline buffers, dopamine oxidation induces the formation of submicrometric particles of PDA. To obtain submicrometric clusters of USPIO, we hypothesized that USPIO coated with dopamine (USPIO@Dopamine) would be incorporated as building blocks during the formation of PDA particles. Thus, we synthetized USPIO by a classical co-precipitation method using a 2:1 FeCl₃:FeCl₂ ratio. After extensive washing steps, purified USPIO were incubated with dopamine in pure water for 15 minutes under continuous sonication to obtain USPIO@dopamine. The resulting solution containing USPIO@Dopamine and free dopamine was stirred using a mechanical disperser at room temperature and ammonia was added to start dopamine polymerization. This led to the self-assembly of USPIO₍ₙ₎@PDA submicrometric clusters. The mean hydrodynamic diameters of the USPIO₍ₙ₎@PDA ranged from 300 nm to 700 nm depending on the concentration of ammonia during cluster formation as measured by dynamic light scattering, with polydispersity indexes <0.2 (FIGS. 1 and 2) and zeta-potentials ranging between −37 and −42 mV. The particles produced were large enough to be rapidly separated from an aqueous solution using a bench magnet.

### Physical Characterization of USPIO₍ₙ₎@PDA Submicrometric Clusters

Using a preclinical MRI, at room temperature and 7T (300 MHz), the relaxivity values were, for r1, r2 and r2* were respectively 0.35, 139.9 and 301.7 mM⁻¹·s⁻¹. All these parameters are within the range of previously reported value using USPIO with similar crystallite sizes, supporting that clustering USPIO using PDA preserves their favorable superparamagnetic properties.

### Biodegradability of USPIO₍ₙ₎@PDA

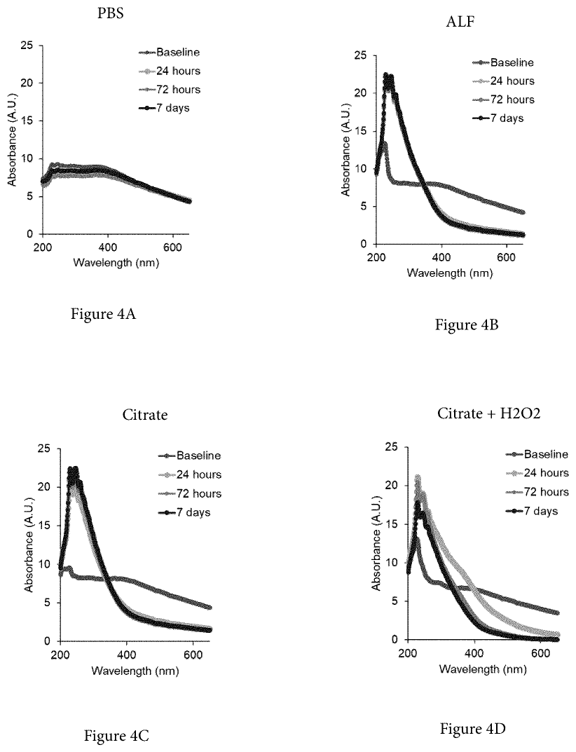

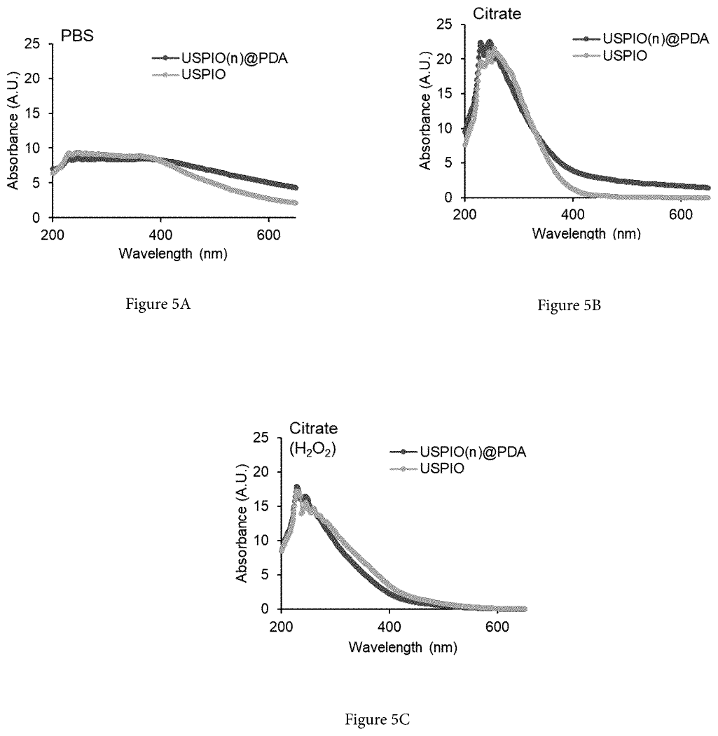

Biodegradability of 700 nm USPIO₍ₙ₎@PDA was investigated both in vitro and in vivo. First, the particles were incubated at 37° C. in phosphate buffered saline (PBS), artificial lysosomal fluid (ALF), citrate buffer or citrate buffer with hydrogen peroxide. These buffers mimic the lysosomal environment where nanoparticles accumulate after intravenous injection. Degradation was monitored by direct visual inspection and ultraviolet-visible spectroscopy (UV-Vis) during 1 week at 37° C. under mild agitation FIG. 4). Visual aspect and UV-vis spectroscopy of USPIO@Dopamine and USPIO₍ₙ₎@PDA in PBS did not change significantly during the monitoring. In ALF and citrate buffers, the brown color of the USPIO@Dopamine solution turned with time into the expected yellow color of free Fe(III) ions. The same phenomenon seemed to occur for USPIO₍ₙ₎@PDA solutions but an additional dark precipitate remained present at every time points. This precipitate appeared dark-brown on bright field microscopy and was not attracted by a magnet. This appearance was compatible with free PDA remaining after degradation of the USPIO. In line with this hypothesis, this precipitate progressively solubilized in samples containing hydrogen peroxide, which generates hydroxyl radicals that are able to degrade PDA. UV-vis spectroscopy further supported these findings by showing progressive degradation of USPIO₍ₙ₎@PDA in ALF and citrate, with persistence of a chemical specie absorbing in the near infrared region (650 nm), compatible with PDA (FIG. 5). In citrate with hydrogen peroxide buffer, the absorbance of the solution initially containing USPIO₍ₙ₎@PDA progressively diminished in the near-infrared region until reaching zero after 7 days of incubation. At day 7, the UV-vis curve was almost undistinguishable between solutions initially containing USPIO@Dopamine or USPIO₍ₙ₎@PDA, supporting complete degradation of PDA in this buffer.

Second, the degradation of USPIO₍ₙ₎@PDA was investigated in cell culture of macrophages, the cell type in which large particles accumulate after intravenous injection in vivo. Macrophages were obtained by activation of a human monocytic cell line (THP-1) and were incubated with either USPIO(n)@PDA or non-biodegradable commercial MPIO (Dynabeads MyOne) made of USPIO embedded in a polystyrene matrix. After 96 hours, the cells and particles were observed by transmission electronic microscopy. Both types of particles were internalized at this time point. Whereas commercial MPIO remained morphologically intact, USPIO(n)@PDA fragmented into smaller particles, demonstrating that the PDA matrix is rapidly degraded and releases USPIO once internalized in macrophages.

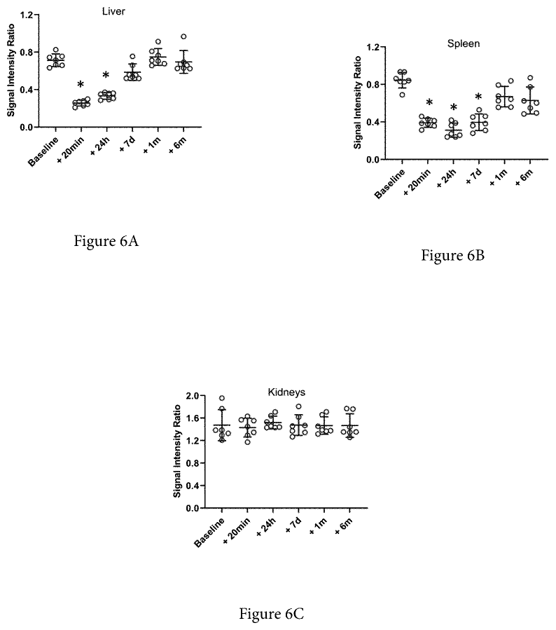

In vivo, the degradation of USPIO₍ₙ₎@PDA was investigated in mice at different time points after intravenous injection (from 1 hour to 6 months) by MRI (FIG. 6) and histochemistry with Prussian blue staining. All the methods concur in showing that USPIO₍ₙ₎@PDA first accumulate in the liver and spleen (reticulo-endothelial system) and that their iron oxide and superparamagnetic components are subsequently degraded. No residual particles were detected more than 30 days after intravenous injection. Importantly, there was no significant accumulation of USPIO₍ₙ₎@PDA in the kidneys, lungs or heart at any time points. No microvascular plugging was observed.

### Biocompatibility of USPIO₍ₙ₎@PDA

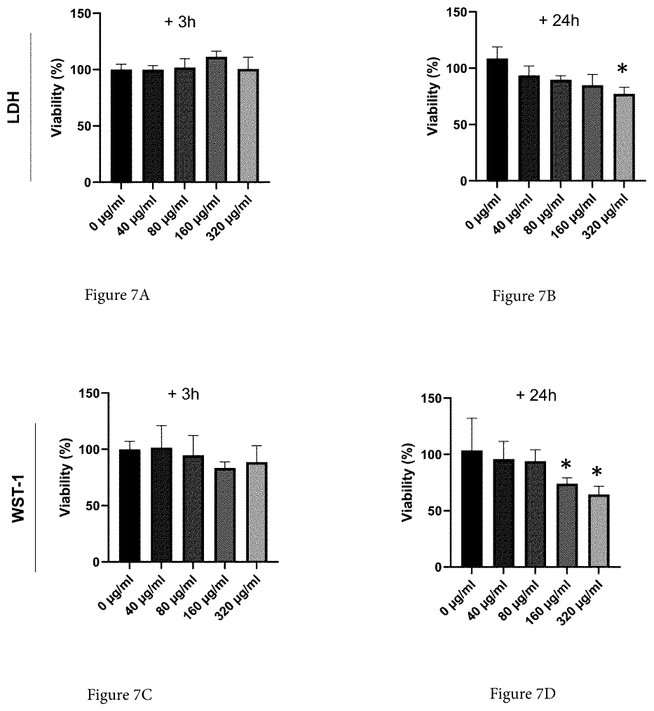

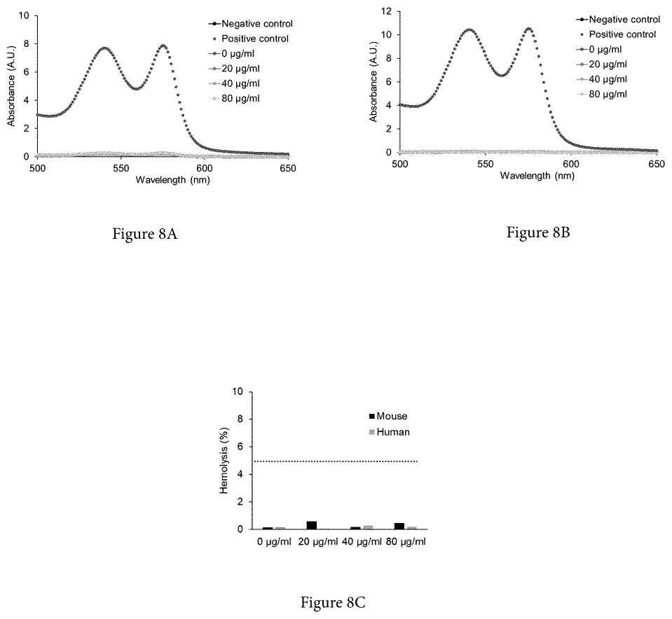

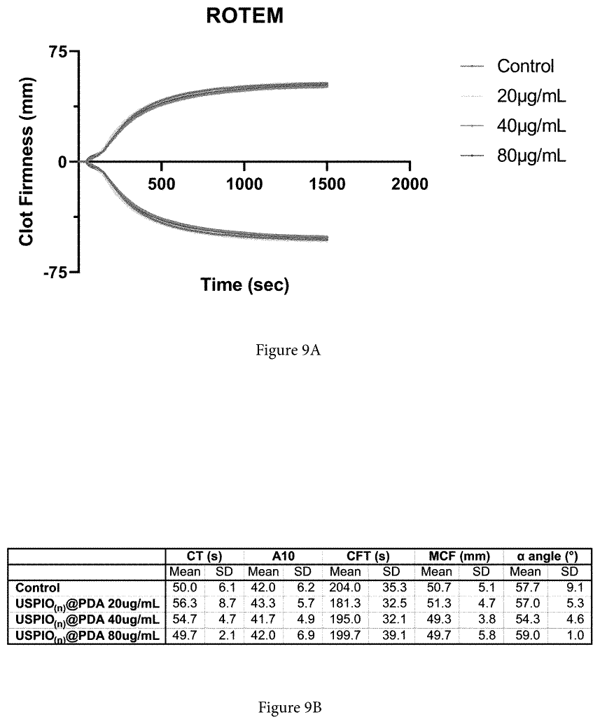

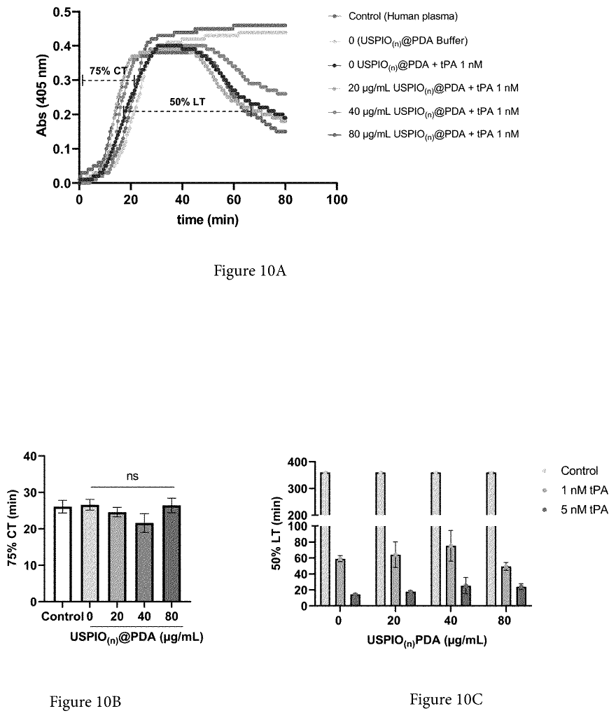

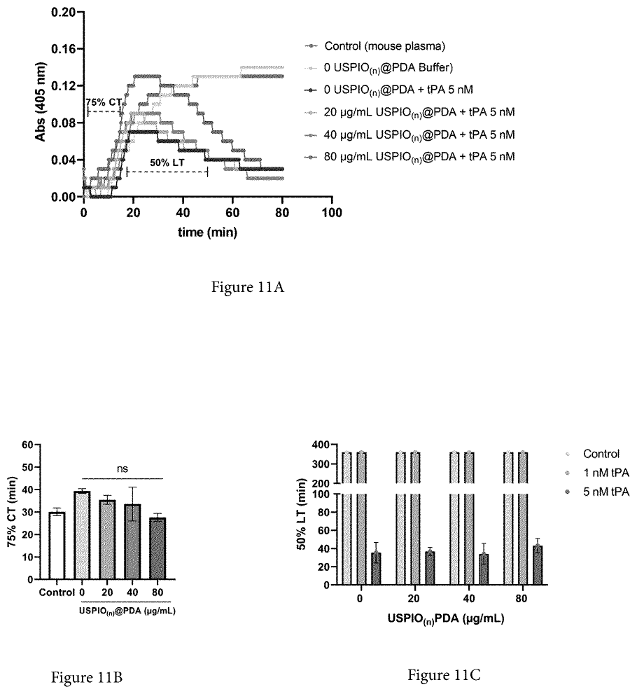

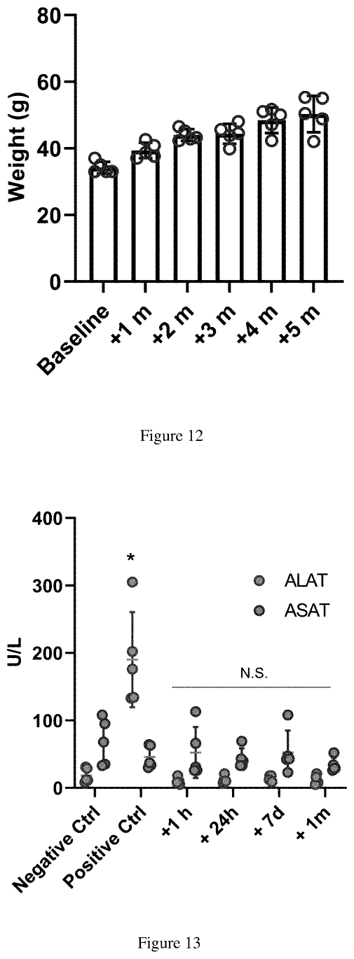

The biocompatibility of USPIO₍ₙ₎@PDA was investigated both in vitro and in vivo. No significant cytotoxicity on endothelial cells (HUVEC) was detected at doses up to 320 g/ml for 3 hours (FIG. 7). At 24 hours, the viability of endothelial cells was slightly reduced in the high-dose groups. Hemolysis (FIG. 8), coagulation (FIG. 9) and fibrinolysis (FIGS. 10 and 11) were all unaffected by USPIO₍ₙ₎@PDA using either human or murine blood. After intravenous injection of 4 mg/kg (equivalent iron) of USPIO₍ₙ₎@PDA, body weight remained within normal ranges (FIG. 12) and no liver enzymes (FIG. 13), inflammatory cytokines (FIG. 14) nor histological findings were of toxicological significance.

### Conjugation of USPIO₍ₙ₎@PDA to Monoclonal Antibodies for Targeted Imaging

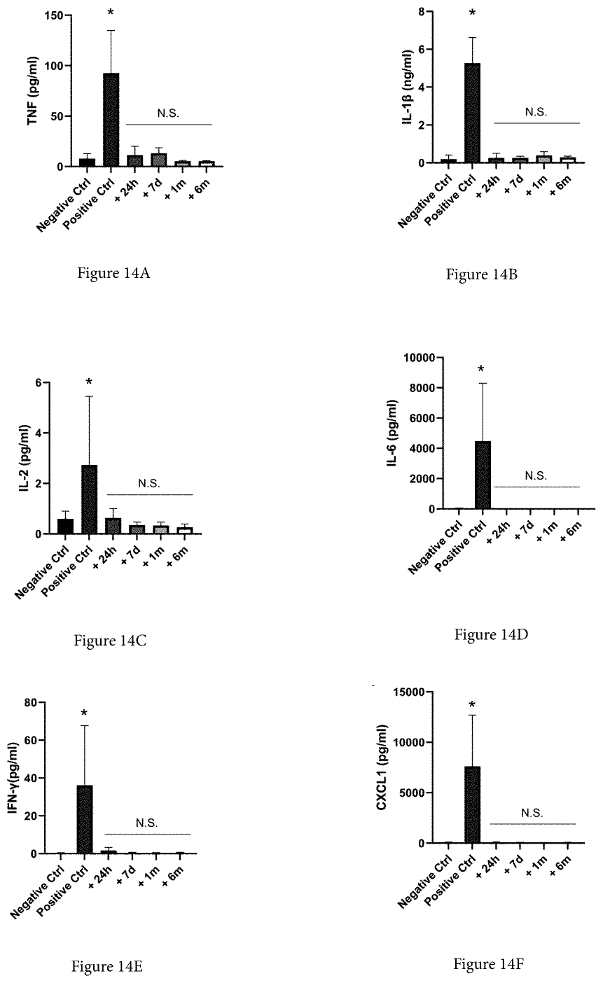

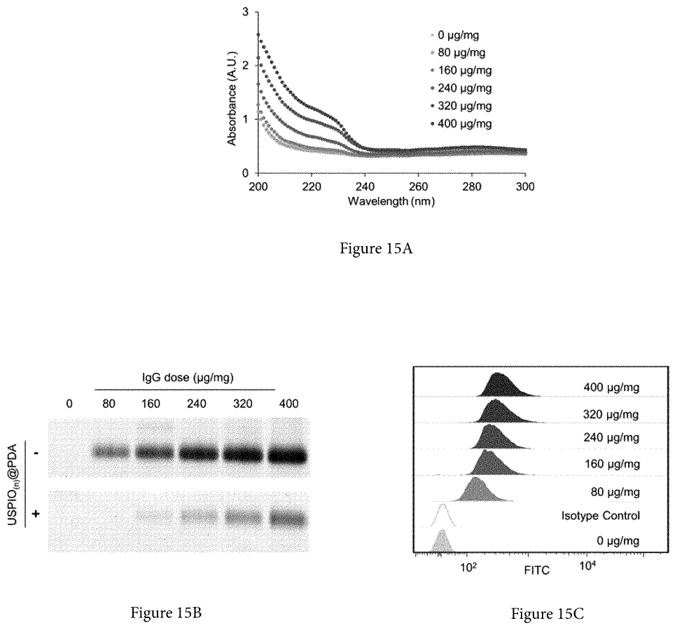

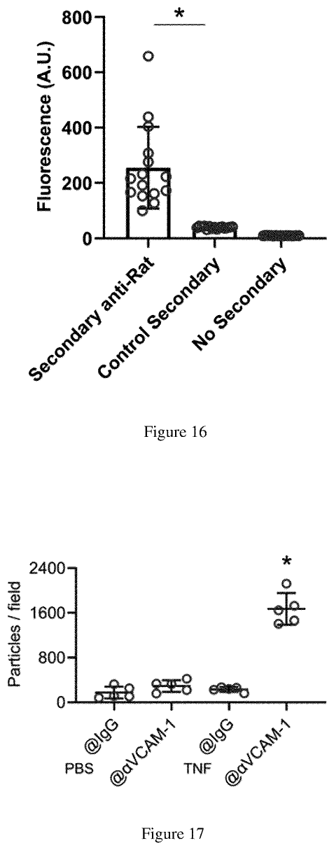

Having demonstrated favorable biocompatibility and biodegradability profiles, we investigated the feasibility of using 700 nm USPIO₍ₙ₎@PDA submicrometric clusters as a platform for molecular imaging. To this aim, we coated USPIO₍ₙ₎@PDA with antibodies in phosphate buffer at pH 8.5 since alkaline buffer favors reactive quinone over catechol groups on the PDA coating. We performed a dose-response experiment by varying the concentration of antibodies in the solution during coupling. Then, we measured the concentration of bound antibodies on USPIO₍ₙ₎@PDA by flow cytometry and the concentration of remaining antibodies in the solution by SDS-PAGE and UV-Vis (FIG. 15). We selected the dose of 160 μg of antibodies for 1 mg of USPIO₍ₙ₎@PDA for further experiments, since higher doses did not significantly increase the concentration of bound antibodies. Using this ratio, we coated USPIO₍ₙ₎@PDA with monoclonal antibodies targeting mouse vascular-cell adhesion molecule 1 (VCAM-1), mucosal addressin cell adhesion molecule 1 (MAdCAM-1) or control immunoglobulin G (USPIO₍ₙ₎@PDA@αVCAM-1, USPIO₍ₙ₎@PDA@αMAdCAM-1 and USPIO₍ₙ₎@PDA@IgG, respectively). Using fluorescent secondary antibodies, the conjugated USPIO₍ₙ₎@PDA clusters can be detected by immunofluorescence by revealing the primary antibodies on their surface (FIG. 16).

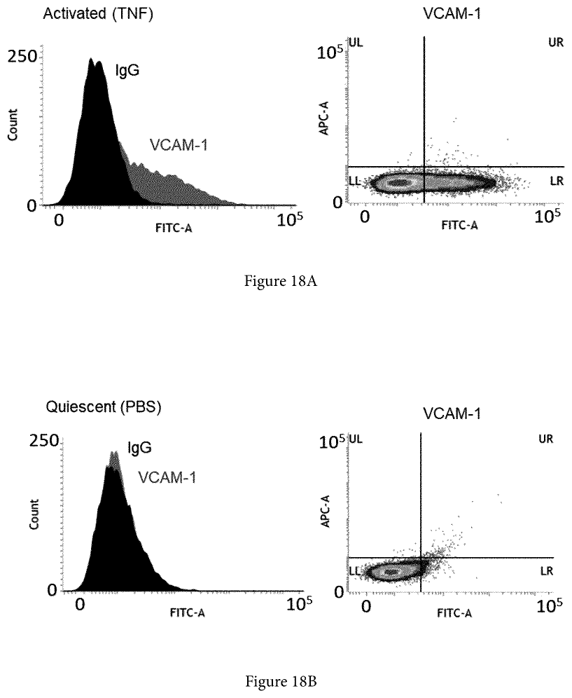

Then, we investigated the binding of targeted USPIO@PDA in vitro. To this aim, anti-human USPIO₍ₙ₎@PDA@αVCAM-1 or control USPIO₍ₙ₎@PDA@IgG were incubated with either quiescent or activated cerebral endothelial cells (hCMECD/3). The number of bound particles was evaluated by immunofluorescence microscopy. USPIO₍ₙ₎@PDA@αVCAM-1 bound significantly more to activated endothelial cells than control USPIO₍ₙ₎@PDA@IgG (FIG. 17). Moreover, USPIO₍ₙ₎@PDA@αVCAM-1 bound significantly more to activated than quiescent endothelial cells, in line with a higher expression of VCAM-1 by the formers (FIG. 18). These results demonstrate that USPIO@PDA coated with anti-VCAM-1 monoclonal antibodies are able to bind selectively activated endothelial cells.

### USPIO₍ₙ₎@PDA@αVCAM-1 Reveal Neuroinflammation at High Sensibility

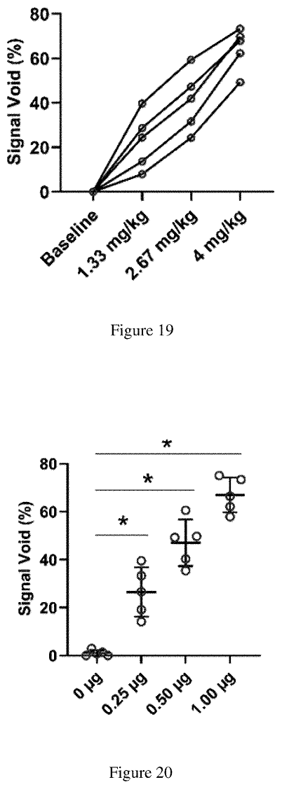

To determine the feasibility of molecular MRI using targeted USPIO(n)@PDA, we used an experimental model of neuroinflammation, induced by intrastriatal injection of E. coli lipopolysaccharide (LPS). Twenty-four hours after intrastriatal injection of LPS (1.0 μg), brain MRI was performed both before and 3 minutes after iterative injections of USPIO₍ₙ₎@PDA@αVCAM-1 corresponding to doses from 1.33 to 4 mg/kg of iron (FIG. 19). On pre-contrast images, no significant difference between the right and left hemispheres was observed in these mice on neither T2 nor T2* weighted images. After intravenous injection of USPIO₍ₙ₎@PDA@αVCAM-1, numerous signal void were observed predominantly in the right striatum. Iterative injection demonstrated that higher doses led to more signal voids. We selected the 4 mg/kg dose for further experiments to achieve high sensitivity while keeping within clinically tolerated doses of iron. Notably, USPIO₍ₙ₎@PDA@αVCAM-1 were detectable at clinically relevant spatial resolution in this model and at this dose.

Thereafter, to investigate whether the signal voids of USPIO₍ₙ₎@PDA@αVCAM-1 correlate with the severity of neuroinflammation, we administered different doses of LPS (0, 0.25, 0.5 or 1.0 μg) in the right striatum of naive mice. Twenty-four hours thereafter, we injected 4 mg/kg of USPIO₍ₙ₎@PDA@αVCAM-1 intravenously and performed post-contrast T2*-weighted MRI of the brain. Consistent with a higher expression of VCAM-1, more signal voids were observed in the right hemisphere of the mice that received the highest doses of LPS (FIG. 20). Importantly, almost no signal void was observed in the brain of control mice, in line with a low basal expression of VCAM-1. Moreover, by comparing post to pre-injection images, we were able to generate 3D maps of VCAM-1 expression in the brain in vivo and at high spatial resolution.

USPIO₍ₙ₎@PDA@αVCAM-1 Combines High Sensitivity with High Specificity

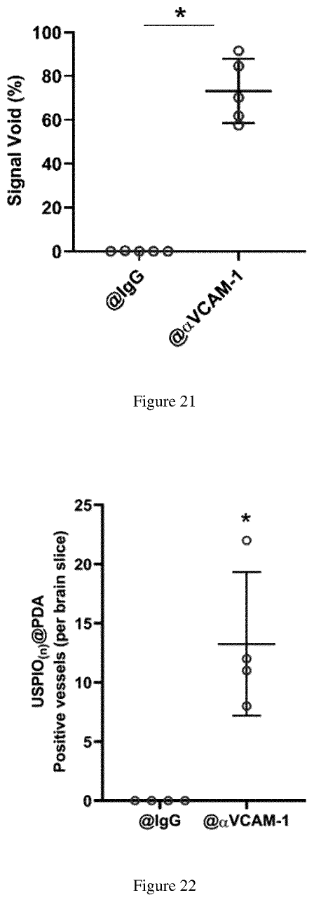

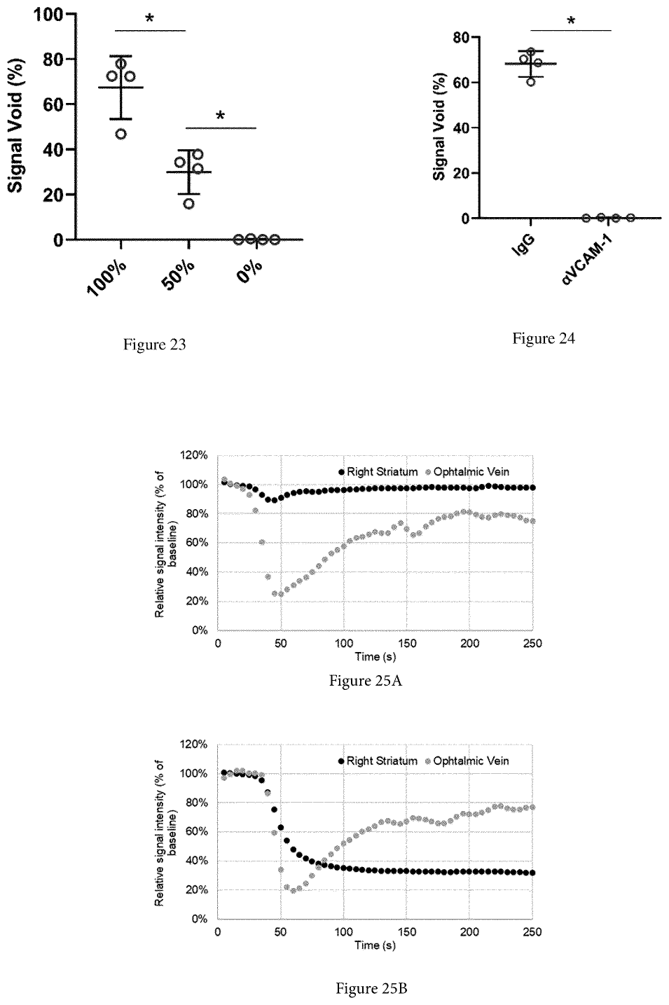

To investigate the specificity of our method, we compared USPIO₍ₙ₎@PDA@αVCAM-1 to control USPIO₍ₙ₎@PDA@IgG. Whereas USPIO₍ₙ₎@PDA@αVCAM-1 induced numerous signal voids in the right striatum 24 hours after intrastriatal injection of LPS (1 μg), no signal void was visible in mice that received USPIO₍ₙ₎@PDA@IgG (FIG. 21). Histological analyses confirmed the MRI findings by revealing significantly more USPIO₍ₙ₎@PDA@αVCAM-1 than USPIO₍ₙ₎@PDA@IgG in the right hemisphere of LPS treated mice (FIG. 22). By varying the ratio of IgG and αVCAM-1 on the surface of particles, we observed that the higher the concentration of targeted antibodies, the higher the binding of USPIO₍ₙ₎@PDA@αVCAM-1 in the inflamed striatum (FIG. 23). Moreover, when the mice were pre-treated with αVCAM-1 monoclonal antibodies to saturate USPIO₍ₙ₎@PDA@αVCAM-1 binding sites, no significant binding of USPIO₍ₙ₎@PDA@αVCAM-1 was observed, confirming the specificity of the contrast agent (FIG. 24).

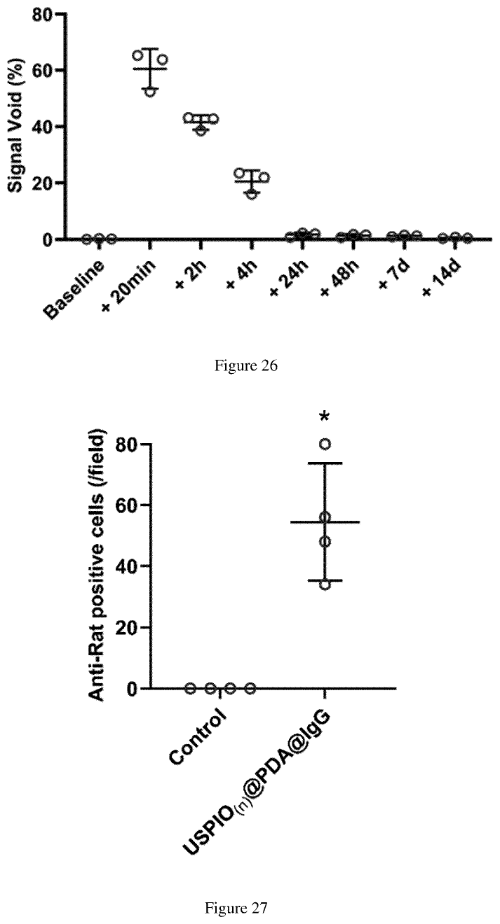

Using high temporal resolution imaging, we also investigated the kinetic of USPIO₍ₙ₎@PDA@αVCAM-1 binding on activated endothelial cells in the LPS model. As shown on FIG. 25, the targeted particles rapidly accumulate in the right striatum, with near maximal contrast reached less than 60 seconds after intravenous injection. Control USPIO(n)@PDA@IgG only induced a short transient drop of T2*-weighted signal in the right striatum, consistent with a lack of binding. Importantly, both particles were rapidly cleared from the circulation with an estimated primary half-life of 50-70 seconds. At later time-points, T2*-weighted imaging revealed progressive unbinding of the particles from the right striatum with near complete disappearance of USPIO₍ₙ₎@PDA@αVCAM-1 induced signal voids 24 hours after injection (FIG. 26). Histological analyses revealed that unbound USPIO(n)@PDA@IgG accumulated in macrophages in the liver (FIG. 27).

Altogether, these experiments demonstrate that USPIO₍ₙ₎@PDA@αVCAM-1 are both sensitive and specific to reveal VCAM-1 overexpression in the brain vasculature.

Clustering USPIO into Large Submicrometric Clusters Improves the Sensitivity of Molecular Imaging

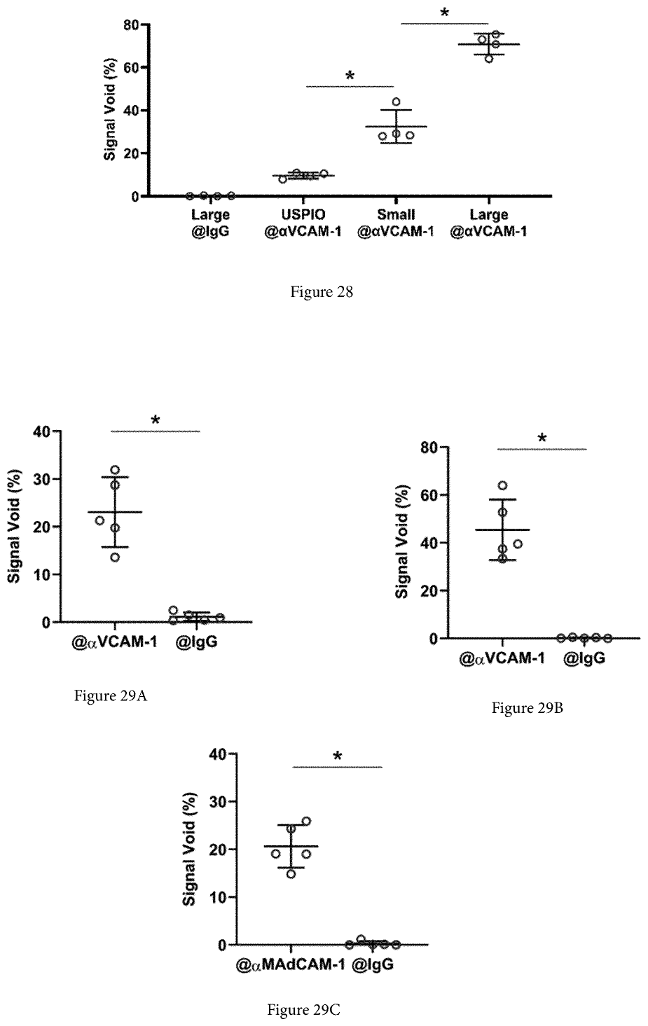

To illustrate the gain in sensitivity provided by clustering USPIO into submicrometric particles, we compared the sensitivity of unclustered USPIO, small (300 nm) and large (700 nm) USPIO₍ₙ₎@PDA clusters conjugated to anti-VCAM-1 monoclonal antibodies to reveal endothelial activation. In the LPS model of neuroinflammation, mice received 4 mg/kg of either particles and MRI was performed 20 minutes thereafter (to allow clearance of the smallest particles). Quantitative analysis revealed significantly more signal void in the mice that received the largest particles (FIG. 28). Almost no signal void was observed in mice that received control large USPIO₍ₙ₎@PDA@IgG. These results support the improved sensibility of large submicrometric particles compared to unclustered USPIO for molecular imaging of VCAM-1.

### USPIO₍ₙ₎@PDA Conjugated to Antibodies Targeting Activated Endothelial Cells Reveal Inflammation in Clinically Relevant Experimental Models

Then, we performed molecular imaging of endothelial activation in more clinically relevant experimental models. First, in a model of ischemic stroke induced by permanent occlusion of the middle cerebral artery (pMCAo). In this model, aseptic inflammation develops in the subacute phase (from 24 hours to 7 days after pMCAo), which is thought to play a key role in stroke pathophysiology. At 24 hours after pMCAo, intravenous injection of USPIO₍ₙ₎@PDA@αVCAM-1 induced numerous signal voids in the right hemisphere, in the periphery of the ischemic lesion (FIG. 29a). In contrast, no significant signal void was observed after injection of control USPIO₍ₙ₎@PDA@IgG. These data demonstrate that USPIO₍ₙ₎@PDA@αVCAM-1 can unmask the neuroinflammatory reaction taking place in the subacute phase of ischemic stroke.

Second, in a model of acute kidney injury induced by rhabdomyolysis. In this model, an intramuscular injection of glycerol is performed in the two limbs to induce rhabdomyolysis, thereby releasing myoglobin from the muscles into the bloodstream and leading to subsequent acute kidney injury related to hypovolemia and direct toxicity of myoglobin on renal tubules. In this model, USPIO₍ₙ₎@PDA@αVCAM-1 revealed endothelial inflammation mainly in the kidney medulla, in line with anatomical repartition of renal tubules (FIG. 29b). Again, USPIO₍ₙ₎@PDA@IgG did not induce any significant signal void.

Lastly, in a model of inflammatory bowel disease. Mice were fed during 5 days with dextran sulfate sodium (DSS) in the drinking water. DSS induces intestinal inflammation by disrupting the intestinal epithelial monolayer lining, leading to the entry of luminal bacteria and associated antigens into the mucosa, triggering an immune response. After 2 days without DSS, we performed molecular imaging of the descending colon both before and after injection of USPIO₍ₙ₎@PDA@MAdCAM-1, targeted to an adhesion molecule overexpressed by activated endothelial cells in mucosal tissues. As shown on FIG. 29c, intravenous injection of USPIO₍ₙ₎@PDA@MAdCAM-1 induced numerous signal void in the inflamed mucosa of DSS-treated mice, whereas USPIO@PDA@IgG did not.

Altogether, these results demonstrate that immuno-MRI using targeted USPIO₍ₙ₎@PDA can reveal inflammation in clinically relevant experimental models, as shown in three different organs (brain, kidney and intestines) and with two different targets (VCAM-1 and MAdCAM-1).

1. A particle having a hydrodynamic diameter comprised between 200 and 2000 nm, said particle comprising ultrasmall particles of iron oxide having a diameter between 1 and 50 nm embedded within a polymer matrix selected from polycathecolamines or polyserotonine.

2. The particle according to claim 1, wherein the iron oxide is selected from Fe₂O₃, Fe₃O₄, or a mixture of Fe₂O₃ and Fe₃O₄.

3. The particle according to claim 1, wherein the polymer matrix is from polydopamine (PDA), polynorepinephrine (PNE), polyepinephrine (PEP) and polyserotonine.

4. The particle according to claim 1, wherein the iron concentration is comprised between 50% and 95% in weight with respect to the to total weight of the particle.

5. A suspension of particles according to claim 1.

6. A process for preparing a suspension of particles according to claim 5, comprising the steps of:
a) Preparing a suspension of ultrasmall particles of iron oxide having a diameter between 1 and 50 nm;
b) Coating of the ultrasmall particles of iron oxide with a catecholamine or serotonine;
c) Polymerizing the catecholamine or serotonine in the presence of the ultrasmall particles of iron oxide,
d) Terminating said polymerization; and
e) Recovering a suspension of particles.

7. Suspension of particles or particle obtained by the process according to claim 6.

8. Conjugate comprising a particle according to claim 1 and a molecule comprising free amine or thiol groups.

9. Conjugate according to claim 8, wherein the molecule comprising free amine or thiol groups is chosen from a protein, a peptide, a nanobody, a monoclonal antibody or a molecule comprising a radiolabeled metal.

10. Conjugate according to claim 9, wherein the monoclonal antibody is chosen from vascular-cell adhesion molecule 1 (VCAM-1), intercellular adhesion molecule 1 (ICAM-1), P-Selectin, E-Selectin or mucosal addressin cell adhesion molecule 1 (MAdCAM-1).

11. An in vivo method of imaging comprising the step of administering to a patient an effective amount of the particle according to claim 1.

12. The method according to claim 11, wherein the method of imaging is selected from Magnetic Resonance Imaging (MRI), Magnetic Particle Imaging (MPI), photoacoustic imaging and Positron Emission Tomography (PET).

13. A composition comprising a suspension of particles according to claim 5.

14. A method of imaging, comprising the step of administering to a patient an effective amount of the composition of claim 13 and an imaging step.

15. (canceled)

16. Conjugate according to claim 8, wherein the molecule comprising free amine or thiol groups is a monoclonal antibody.

17. Conjugate according to claim 8, wherein the molecule comprising free amine or thiol groups is a protein that can be a tissue plasminogen activator (tPA) or a fragment thereof.

18. Conjugate according to claim 8, wherein the molecule comprising free amine or thiol groups is a recombinant tissue plasminogen activator (rtPA).

19. Conjugate according to claim 8, wherein the molecule comprising free amine or thiol groups is alteplase, reteplase or tenecteplase.

20. Conjugate comprising a suspension of particles according to claim 5 and a molecule comprising free amine or thiol groups.

21. An in vivo method of imaging comprising the step of administering to a patient an effective amount of the conjugate according to claim 8 and an imaging step.
